(19) United States
(12) Patent Application Publication
(10) Pub. No.: US 2015/0164679 A1
(43) Pub. Date: Jun. 18, 2015

(54) OSTOMY DEVICES

(71) Applicant: Mayo Foundation for Medical Education and Research, Rochester, MN (US)

(72) Inventors: Laurie J. Maidl, Rochester, MN (US); William A. Faubion, Rochester, MN (US); Eric J. Dozois, Rochester, MN (US); Troy J. Ziegler, Plymouth, MN (US)

(21) Appl. No.: 14/412,127

(22) PCT Filed: Jul. 1, 2013
(86) PCT No.: PCT/US2013/048953

§ 371 Date: Dec. 30, 2014

Related U.S. Application Data
(60) Provisional application No. 61/667,762, filed on Jul. 3, 2012.

(51) Int. Cl. A61F 5/445; A61F 5/44

(52) U.S. Cl. CPC A61F 5/445; A61F 5/4404; A61F 2005/4455

(57) ABSTRACT

This document provides methods and materials related to ostomy devices. For example, methods and materials that can be used to prevent waste material and/or gas from escaping a waste reservoir of an ostomy patient such as ostomy plug devices configured to include a sealing element, a tensioning element, and a cap element are provided.

### CROSS-REFERENCE TO RELATED APPLICATIONS

This application claims the benefit of U.S. Provisional Application Ser. 61/667,762, filed Jul. 3, 2012. The disclosure of the prior applications are considered part of (and are incorporated by reference in) the disclosure of this application.

### BACKGROUND

1. Technical Field

This document relates to ostomy devices. For example, this document relates to plug devices for an ostomy (e.g., a colostomy, ileostomy, or urostomy).

2. Background Information

Treating some diseases of the digestive or urinary systems can involve removing all or part of a patient's small intestine, colon, rectum, or bladder. In these cases, waste must be rerouted to exit the body of the subject. The rerouting surgery, known as an ostomy, can involve creating an opening in the abdomen wall so that a portion of the intestinal tract can be brought out to the skin level, resulting in what is called a stoma. Three common types of abdominal wall stomas result from, and may be classified as, a colostomy, ileostomy, and urostomy, which involve patients who have had surgery on their the large intestine, small intestine, and urinary bladder, respectively. Typically, a medical prosthetic known as an ostomy pouching system can be used to collect waste from a diverted biological system as it exits a stoma.

### SUMMARY

This document provides methods and materials related to ostomy devices. For example, this document provides plug devices for an ostomy (e.g., a colostomy, ileostomy, or urostomy).

As described herein, an ostomy plug device can be configured to include a sealing element, a tensioning element, and a cap element. The sealing element can be positioned within a waste collection reservoir of an ostomy patient. The tensioning element can be connected to the sealing element and the cap element in a manner that allows the tensioning element to provide sufficient tension on the sealing element. In some cases, the tension applied to the sealing element from the tensioning element can be sufficient to prevent waste material and/or gas from exiting a waste collection reservoir, while applying minimal pressure against a stricture. The cap element can be configured to be located outside of the ostomy patient. Such cap elements can be configured to cover a stoma and to provide an anchoring structure for a tensioning element such that the tensioning element can provide sufficient tension to the sealing element. In some cases, a tensioning element of an ostomy plug device provided herein can be a solid cord or string and can lack a fluid or gas conduit. In some cases, an ostomy plug device provided herein can be configured to lack fluid and gas conduits that extend from outside a patient to a sealing element located within the patient. In some cases, when the sealing element is inflatable, an ostomy plug device can be configured to include one or more fluid and/or gas conduits capable of being used to inflate the inflatable sealing element. For example, a tensioning element can be in the form of a conduit capable of being used to inflate the inflatable sealing element.

In general, one aspect of this document features an ostomy plug device for sealing a stoma from within an ostomy patient. The ostomy plug device comprises, or consists essentially of, a sealing element, a cap element, and a tensioning element attached to the sealing element and the cap element, wherein the cap element is configured to be located outside of the body of the patient, wherein the sealing element is configured to be implanted through the stoma into at least a portion of a waste collection reservoir located within the patient, and wherein the tensioning element is configured to provide tension to the sealing element using the cap element for anchoring, thereby sealing the stoma from within an ostomy patient. The sealing element can be disc shaped. The sealing element can be biased to have a concave surface facing the stoma, wherein the tension applied to the sealing element by the tensioning element can cause the concave surface to evert to a convex surface. The sealing element can flex upon application of the tension. The cap element can define a port. The port can be configured to allow gas, waste material, or both to be removed from the waste collection reservoir. The cap element can be rigid. The tensioning element can be a cord or string. The tensioning element can be a flexible cord or string. The tensioning element can be solid and can lack a lumen. The waste collection reservoir can be a surgically created waste collection reservoir.

In another aspect, this document features a method for sealing a stoma of an ostomy patient with an ostomy plug device comprising a sealing element, a cap element, and a tensioning element attached to the sealing element and the cap element. The method comprises, or consists essentially of, (a) inserting the sealing element through the stoma into at least a portion of a waste collection reservoir located within the patient, and (b) increasing the tension applied by the tensioning element on the sealing element until the cap element is positioned against an outer surface of the patient and the sealing element seals the stoma from within the patient. The sealing element can be disc shaped. The sealing element can be biased to have a concave surface facing the stoma when not under the tension, and wherein the tension applied to the sealing element by the tensioning element can cause the concave surface to evert to a convex surface, thereby sealing the stoma. The sealing element can flex upon application of the tension. The cap element can define a port. The port can be configured to allow gas, waste material, or both to be removed from the waste collection reservoir. The cap element can be rigid. The tensioning element can be a cord or string. The tensioning element can be a flexible cord or string. The tensioning element can be solid and can lack a lumen. The waste collection reservoir can be a surgically created waste collection reservoir.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a side view of an ostomy plug device positioned within respect of an ostomy patient, in accordance with one embodiment provided herein.

FIGS. 2A and 2B are front and side views, respectively, of a sealing element of an ostomy plug device, in accordance with one embodiment provided herein.

FIGS. 2C and 2D are front and side views, respectively, of a sealing element of an ostomy plug device, in accordance with one embodiment provided herein.

FIGS. 2E and 2F are front and side views, respectively, of a sealing element of an ostomy plug device, in accordance with one embodiment provided herein.

FIGS. 2G and 2H are front and side views, respectively, of a sealing element of an ostomy plug device, in accordance with one embodiment provided herein.

FIG. 3A is an elevated side view of a cap element of an ostomy plug device, in accordance with one embodiment provided herein.

FIG. 3B is an elevated side view of a cap element of an ostomy plug device, in accordance with one embodiment provided herein.

FIG. 4 is a side view of an ostomy plug device positioned within respect of an ostomy patient, in accordance with one embodiment provided herein.

### DETAILED DESCRIPTION

This document provides methods and materials related to ostomy devices. For example, this document provides methods and materials that can be used to prevent waste material and/or gas from escaping a waste reservoir of an ostomy patient.

In general, an ostomy plug device provided herein can be configured to include a sealing element, a tensioning element, and a cap element. A sealing element can be configured to be positioned within a waste collection reservoir of an ostomy patient, and a tensioning element can be connected to the sealing element and a cap element in a manner that allows the tensioning element to provide sufficient tension on the sealing element so as to prevent waste material and/or gas from escaping a waste reservoir of an ostomy patient. In some cases, the tension applied to the sealing element from the tensioning element can be sufficient to prevent waste material and/or gas from exiting a waste collection reservoir, while applying minimal pressure against a stricture. The cap element can be configured to be located outside of the ostomy patient. In some cases, a cap element can be configured to cover a stoma and to provide an anchoring structure for a tensioning element such that the tensioning element can provide sufficient tension to the sealing element.

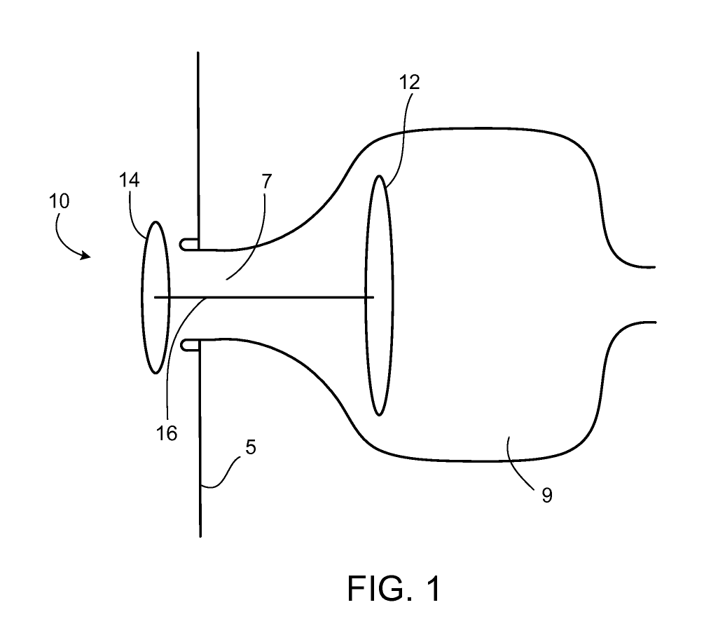

With reference to FIG. 1, an ostomy plug device 10 can include a sealing element 12, a tensioning element 16, and a cap element 14. Tensioning element 16 can be attached to sealing element 12 and cap element 14. In some cases, tensioning element 16 can extend through cap element 14 such that a user can manually adjust the tension applied by tensioning element 16 on sealing element 12. For example, tensioning element 16 can be a cord, string, wire, or conduit that extend from sealing element 12 through cap element 14 such that a user can manually pull on one end of tensioning element 16 to adjust the tension applied by tensioning element 16 on sealing element 12. In some cases, tensioning element 16 can be configured to be flexible such that a predetermined amount of tension is applied to sealing element 12 based on the elasticity of the material of tensioning element 16 and the distance between cap element 14 and sealing element 12.

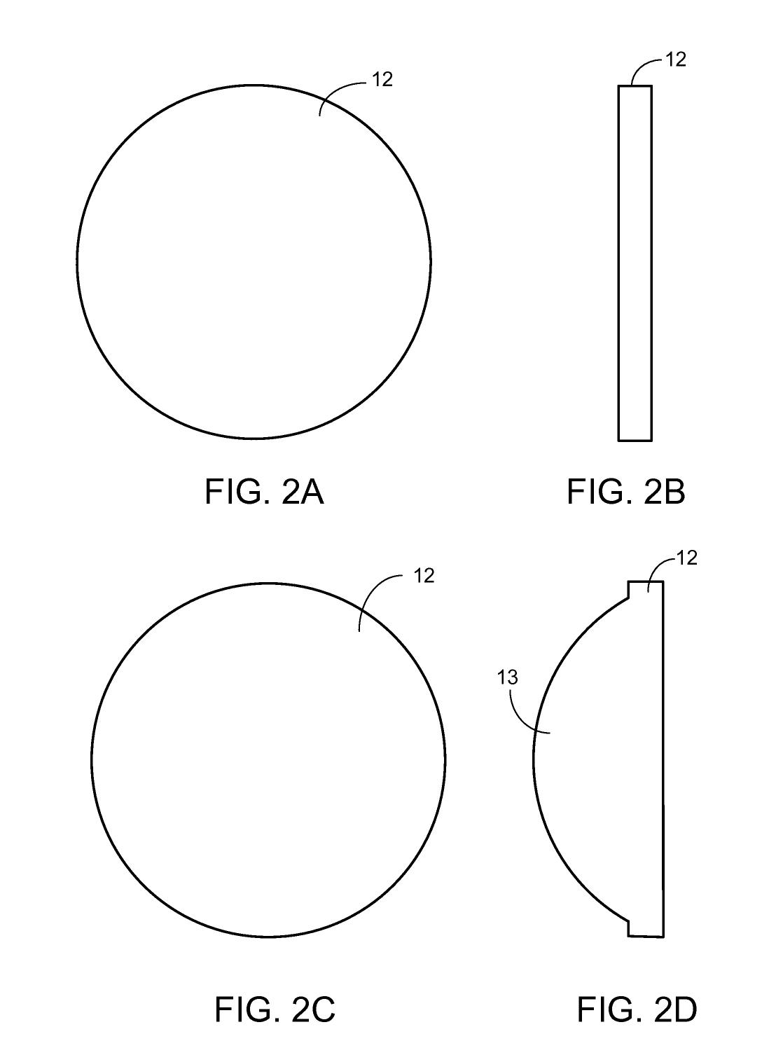

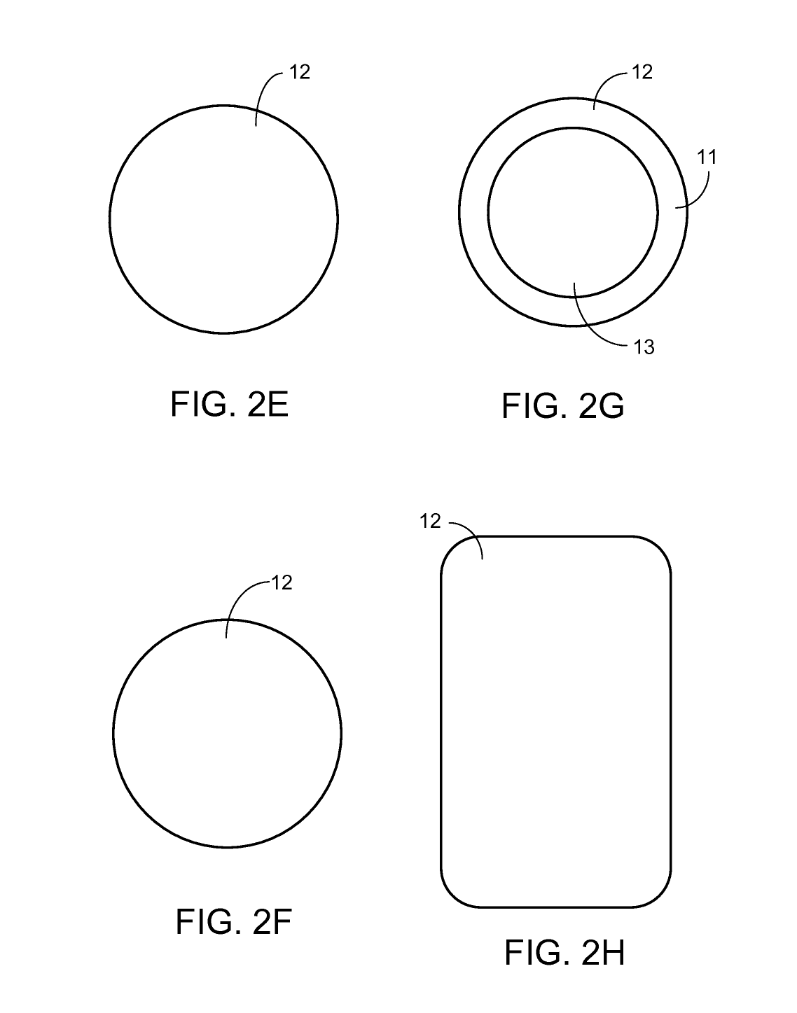

Sealing element 12 can have any appropriate shape and configuration provided that it is capable of preventing waste material and/or gas from exiting a waste collection reservoir 9 (e.g., a surgically created waste collection reservoir) when tension is applied by tensioning element 16. For example, sealing element 12 can be disk shaped as shown in FIGS. 2A and 2B, dome shaped as shown in FIGS. 2C and 2D, ball shaped as shown in FIGS. 2E and 2F, or covered ring shaped (e.g., a donut shape without a hole) as shown in FIGS. 2G and 2H. Other examples of shapes and configurations for a sealing element include, without limitation, bulbous lens, discs, saucers, and caps. As shown in FIG. 2D, a dome shaped sealing element 12 can have one surface 13 that is convex and an opposing surface that is concave or flat with respect to that convex surface. Such a convex surface can be configured to extend into the opening of an ostomy in a manner that helps prevent waste material and/or gas from exiting the waste collection reservoir. As shown in FIG. 2G, a covered ring shaped sealing element 12 can have an outer ring portion 11 and an inner portion 13.

In some cases, sealing element 12 can be inflatable (e.g., gel, fluid, or gas inflatable). For example, a ball shaped sealing element 12 as shown in FIGS. 2E and 2F or a covered ring shaped (e.g., a donut shape without a hole) sealing element 12 as shown in FIGS. 2G and 2H can be inflatable. In such cases, a conduit can extend from sealing element 12 to the outside of an ostomy patient such that sealing element 12 can be inflated and deflated as needed. In some cases, tensioning element 16 can be configured as a conduit for inflating and deflating an inflatable sealing element.

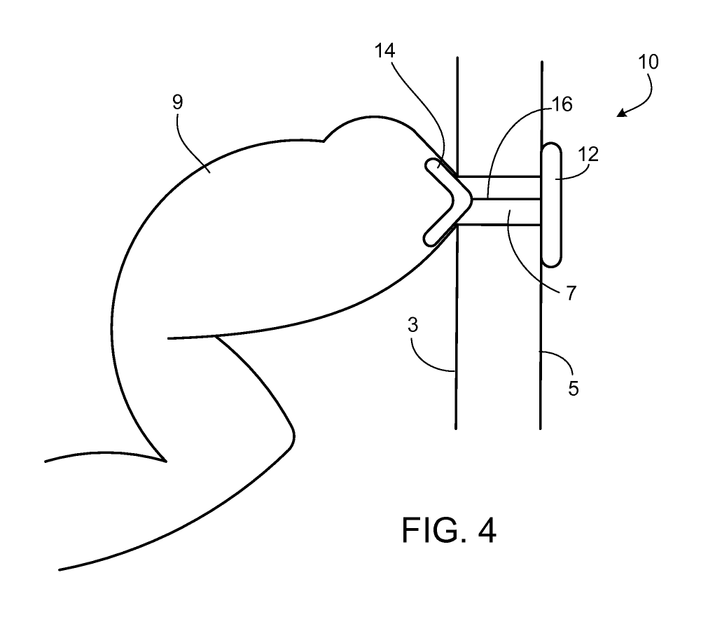

Sealing element 12 can be flexible. For example, a disk shaped sealing element 12 as shown in FIGS. 2A and 2B can be flexible such that when tension is applied by tensioning element the disk shaped sealing element 12 flexes in a direction as shown, for example, in FIG. 4. Release of the tension by tensioning element 16 can allow the disk shaped sealing element 12 to return to its normal disk configuration. In some cases, the application of tension can be used when a user intends to prevent waste material and/or gas from exiting the waste collection reservoir, and the removal of tension can be used when the user intends to remove waste material and/or gas from the waste collection reservoir. In some cases, a user can completely remove sealing element 12 to remove waste material and/or gas from the waste collection reservoir.

In some cases, sealing element 12 can be a convex/concave everting structure. For example, sealing element 12 can be biased to have a concave surface facing the opening of an ostomy when sealing element 12 is under little or no tension from tensioning element 16. The application of tension from tensioning element 16 can cause sealing element 12 to evert such that the concave surface becomes a convex surface and forms a seal with an inner lining of the ostomy patient's waste collection reservoir. In such cases, release of at least some of the tension applied to sealing element 12 can allow sealing element 12 to evert back to its naturally biased configuration such that the surface facing the opening of the ostomy is a concave surface and the seal between sealing element 12 and an inner lining of the ostomy patient's waste collection reservoir is opened, thereby allowing waste material and/or gas to be removed from the waste collection reservoir.

Sealing element 12 can be made of any appropriate material. For example, sealing element 12 can be made of a biocompatible material such as a plastic, a polymer of a natural material, or a polymer of a synthetic material. In some cases, sealing element 12 can be soft and compliant in a manner that allows it to be easily inserted through a stricture 7 and can be rigid enough to prevent it from collapsing and/or breaking its seal. Sealing element 12 can have a large enough surface area to create an effective seal with minimal pressure against stricture 7. In some cases, a surface of sealing element 12 (e.g., a surface configured to engage an inner surface of the waste collection reservoir) can include tacky or sticky material. Such material can be in the form of a coating.

In some cases, a catheter, rod, or other device (e.g., a fenestrated obturator) can be advanced within a stoma to release a seal formed between sealing element 12 and an inner lining of the ostomy patient's waste collection reservoir. For example, to release a seal formed between sealing element 12 and an inner lining of the ostomy patient's waste collection reservoir, the tension applied to sealing element 12 by tensioning element 16 can be release and a catheter can be advanced into the stoma so as to push sealing element 12 away from the inner lining of the ostomy patient's waste collection reservoir. In such cases, the catheter can be used to remove waste material and/or gas from the waste collection reservoir.

Cap element 14 can have any appropriate shape and configuration provided that it is capable of providing an anchor for tensioning element 16. For example, cap element 14 can be disk shaped, dome shaped, covered ring shaped (e.g., a donut shape without a hole), or donut shaped. Other examples of shapes and configurations for a cap element include, without limitation, bulbous lens, discs, saucers, and caps. Cap element 14 can lack one or more holes or openings or can define one or more holes or openings. For example, cap element 14 can define an opening or port and can be configured to have a closing structure (e.g., a lid or plug) that can be used to close that opening or port to prevent access to the stoma or waste collection reservoir. Such closing structure can be opened or removed to provide access to the stoma or waste collection reservoir. In some cases, cap element 14 can have a valve that can be actuated from a closed position to an open position to provide access to the stoma or waste collection reservoir.

In some cases, cap element 12 can cover the stoma to absorb and/or prevent leakage of mucus or other material from the stoma. In some cases, cap element 12 can include an adhesive material. Such adhesive material can be in the form of a coating and can help prevent movement of cap element 12. Examples of adhesive materials include, without limitation, reactive adhesives of natural origin, reactive adhesives of natural synthetic origin, non-reactive adhesives of natural origin, and non-reactive adhesives of synthetic origin.

In some cases, cap element 12 can include a tension indicator capable of providing a user with an indication of the degree of tension being applied by tensioning element 16 to sealing element 12. In some cases, cap element 12 can include a gas valve capable of being opened to release gas from the stoma and/or waste collection reservoir.

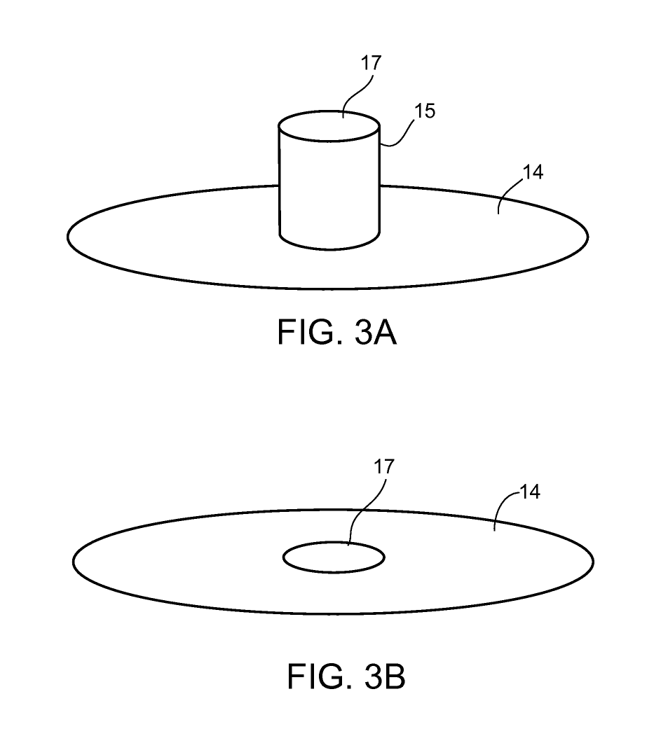

As shown in FIG. 3A, cap element 14 can have a disk shaped base portion and an extended port 15 defining an opening 17. In some cases, extended port 15 can be capable of telescoping away from the base portion. In some cases, cap element 14 can define an opening 17 as shown in FIG. 3B. In some cases, opening 17 can be a port (e.g., flanged port) that allows a device (e.g., an endoscope, camera, or catheter) to be inserted through the port while allowing little or no air to escape.

Cap element 14 can be rigid. For example, a disk shaped cap element 14 as shown in FIG. 3A or 3B can be rigid such that cap element 14 provide an anchoring role when tension is applied sealing element 12 by tensioning element 16.

Cap element 14 can be made of any appropriate material. For example, cap element 14 can be made of a biocompatible material such as a plastic, a polymer of a natural material, or a polymer of a synthetic material.

In some cases, cap element 14 can work in conjunction with sealing element 12 to prevent waste material and/or gas from exiting a waste collection reservoir 9 when tension is applied by tensioning element 16.

With reference to FIG. 4, ostomy plug device 10 can be inserted into an ostomy patient having a surgically created waste collection reservoir 9. Surgically created waste collection reservoir 9 can be formed by surgically combining intestinal material in a manner that results in a reservoir that has a larger diameter and larger volume than a similar length of intestine would naturally have. A stoma provides an exit point for waste material from a waste collection reservoir and can be in the form of a stricture 7. Stricture 7 can be composed of fascia 3 and/or fat and can extend to skin 5. In some cases, stricture 7 can define a stoma surgically created to have a smaller diameter than surgically created waste collection reservoir 9. In some cases, stricture 7 can formed by surgically inducing scar tissue formation to create a stoma having a smaller diameter than surgically created waste collection reservoir 9. In some cases, stricture 7 can include artificially implanted material (e.g., a ring of material such as mesh material) configured to provide rigidity to the stricture.

Ostomy plug device 10 can extend from an internal portion of surgically created waste collection reservoir 9 to outside the ostomy patient. For example, sealing element 14 can be located within an internal portion of surgically created waste collection reservoir 9, cap element 12 can be located along skin 5, and tensioning element 16 can be extend from sealing element 14 to cap element 12. In some cases, ostomy plug device 10 can be located within fascia 3.

In some cases, an ostomy plug device provided herein can be configured to lack fluid and gas conduits that extend from outside an ostomy patient to a sealing element located within the ostomy patient. In some cases, for example when the sealing element is inflatable, an ostomy plug device can be configured to include one or more fluid and/or gas conduits capable of being used to inflate the inflatable sealing element. For example, a tensioning element can be in the form of a conduit capable of being used to inflate an inflatable sealing element.

The materials and methods provided herein can be used by any appropriate ostomy patient or care provider. In some cases, patients or care providers of patients with ileostomy or colostomy can use an ostomy plug device provided herein.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An ostomy plug device for sealing a stoma from within an ostomy patient, wherein said ostomy plug device comprises a sealing element, a cap element, and a tensioning element attached to said sealing element and said cap element, wherein said cap element is configured to be located outside of the body of said patient, wherein said sealing element is configured to be implanted through said stoma into at least a portion of a waste collection reservoir located within said patient, and wherein said tensioning element is configured to provide tension to said sealing element using said cap element for anchoring, thereby sealing said stoma from within an ostomy patient.

2. The ostomy plug device of claim 1, wherein said sealing element is disc shaped.

3. The ostomy plug device of claim 1, wherein said sealing element is biased to have a concave surface facing said stoma, wherein said tension applied to said sealing element by said tensioning element causes said concave surface to evert to a convex surface.

4. The ostomy plug device of claim 1, wherein said sealing element is flexes upon application of said tension.

5. The ostomy plug device of claim 1, wherein said cap element defines a port.

6. The ostomy plug device of claim 5, wherein said port is configured to allow gas, waste material, or both to be removed from said waste collection reservoir.

7. The ostomy plug device of claim 1, wherein said cap element is rigid.

8. The ostomy plug device of claim 1, wherein said tensioning element is a cord or string.

9. The ostomy plug device of claim 1, wherein said tensioning element is a flexible cord or string.

10. The ostomy plug device of claim 1, wherein said tensioning element is solid and lacks a lumen.

11. The ostomy plug device of claim 1, wherein said waste collection reservoir is a surgically created waste collection reservoir.

12. A method for sealing a stoma of an ostomy patient with an ostomy plug device comprising a sealing element, a cap element, and a tensioning element attached to said sealing element and said cap element, wherein said method comprises:
(a) inserting said sealing element through said stoma into at least a portion of a waste collection reservoir located within said patient, and
(b) increasing the tension applied by said tensioning element on said sealing element until said cap element is positioned against an outer surface of said patient and said sealing element seals said stoma from within said patient.

13. The method of claim 12, wherein said sealing element is disc shaped.

14. The method of claim 12, wherein said sealing element is biased to have a concave surface facing said stoma when not under said tension, and wherein said tension applied to said sealing element by said tensioning element causes said concave surface to evert to a convex surface, thereby sealing said stoma.

15. The method of claim 12, wherein said sealing element is flexes upon application of said tension.

16. The method of claim 12, wherein said cap element defines a port.

17. The method of claim 16, wherein said port is configured to allow gas, waste material, or both to be removed from said waste collection reservoir.

18. The method of claim 12, wherein said cap element is rigid.

19. The method of claim 12, wherein said tensioning element is a cord or string.

20. The method of claim 12, wherein said tensioning element is a flexible cord or string.

21. The method of claim 12, wherein said tensioning element is solid and lacks a lumen.

22. The method of claim 12, wherein said waste collection reservoir is a surgically created waste collection reservoir.
